(19) United States
(12) Patent Application Publication
(10) Pub. No.: US 2013/0267919 A1
(43) Pub. Date: Oct. 10, 2013

(54) SOLENOID ACTIVATED VACUUM CONTROL DEVICE

(71) Applicants: RICHARD BRAND CASO, Mission Viejo, CA (US); James Selevan, Laguna Beach, CA (US); Peter Bonin, Newport Beach, CA (US); Irving Quam, Eastvale, CA (US)

(72) Inventors: RICHARD BRAND CASO, Mission Viejo, CA (US); James Selevan, Laguna Beach, CA (US); Peter Bonin, Newport Beach, CA (US); Irving Quam, Eastvale, CA (US)

(21) Appl. No.: 13/440,657

(22) Filed: Apr. 5, 2012

(51) Int. Cl. A61M 1/00

(52) U.S. Cl. CPC A61M 1/0031
USPC 604/319

(57) ABSTRACT

The invention herein applies generally to General-purpose, Surgical and Tracheal suction regulators. In one embodiment of the invention, at least one micro-latching valve is opened and closed by at least one solenoid electromechanical actuator. The micro-latching solenoid valve in turns opens and closes at least one main valve that is connected the hospital vacuum or gas intake conduit. The latching nature of the solenoid, along with its low power activation allows for a battery powered, long-life device. Timing and control of the electromechanical actuator is performed by a low power micro-controller. This provides opportunity for highly accurate timing cycles, user adjustable timing intervals and feedback loop control operations. The invention also features a wireless suction control system of many suction regulators comprising a network. Wherein the plurality of wireless suction regulators are linked to at least one router with at least one wireless transmitter, and the network is linked through the router to the internet.

### FIELD OF THE INVENTION

The present invention relates to the art of suction regulators or vacuum flow control devices.

### STATEMENT REGARDING FEDERALLY-SPONSORED RESEARCH AND DEVELOPMENT

Not applicable.

### BACKGROUND OF THE INVENTION

Suction regulators have been used in hospitals since the 1950s. Dedicated devices or modern regulators designed for specific applications were introduced in the 1960s. The intermittent vacuum regulator was introduced in the 1970s and the combined regulator followed. No significant technological changes have happened since.

Accordingly, hospital and clinic facilities-management systems include vacuum pumps that maintain a negative pressure of −760 millimeters mercury (−1 atmosphere; −14.7 pounds per square inch negative pressure) below atmospheric. Vacuum is defined as the difference between atmospheric pressure and subatmospheric pressure, created by a vacuum-producing device such as a vacuum pump. Suction is defined as the flow of air or fluid, and in some cases solids such as clots and tissue, through suction tubing. Flow rate refers to how fast vacuum pressures draw fluids and air into collection vessel systems during suctioning procedures. Flow is created by lowering the pressure at one end of the tube. Resistance causes reduction in flow rates and prevents maximum flow potential from being achieved.

This “vacuum” is delivered to each bedside via a complex of conduits within the clinic wall structure typically found throughout a hospital or surgery center. At the patient bedside a standard fitting is mounted to the wall or head-board, thereby allowing a regulator to “plug in” to the available vacuum. As different fluids and clinical situations call for different vacuum pressure, a regulator is mounted on the wall to allow for manual adjustment of the vacuum delivered to the patient.

All these devices have relied on the hospital vacuum source as the main power generating engine to control the several mechanical valves to deliver the vacuum either intermittent or continuous. A few manufacturers have made compact, lightweight regulators enclosed in a protective plastic housing; however, some of these devices, are still dependent on the “mechanical” calibration and parts to generate the vacuum pulse.

The main purpose of modern suction regulators is to control suction. Types of suction regulators include:

General-purpose suction regulators used in recovery rooms; in the intensive care and coronary care units; and at the patient's bedside. All of them rely on mechanical control.

Surgical suction regulators are used to control the removal of secretions such as vomitus, mucus, or blood during surgical procedures, as well as secretions in wound cavities after surgery.

Tracheal suction regulators control suctioning that is performed directly or through an endotracheal or tracheostomy tube to clear excess secretions from the trachea or tracheobronchial tree; they are commonly used postoperatively for thoracic surgical patients, postanesthesia patients, and certain intensive care unit patients.

Regulated oral, nasal, and pharyngeal suctioning may be needed to remove excessive secretions from unconscious and/or critically ill patients, as well as from patients recovering from anesthesia. In suctioning, semisolids, liquids, and gases are removed from the stomach and intestinal tract to prevent the buildup of gastric contents and swallowed air.

Intermittent or controlled suctioning is often needed with all the aforementioned regulators to minimize damage to the mucosal lining and blockage of the catheter tip if the tip entraps solids. Too much resistance may compromise the functional efficacy of a suction collection system to the point where potentially life-threatening situations in a clinical setting could occur.

In Thoracic suction regulators produce the vacuum levels and high airflows needed to remove blood, exudate, and air from the pleural cavity, thereby counteracting pneumothorax and allowing the lung to reexpand.

Most human fluids are viscous, thereby requiring significant negative pressure “vacuum” to affect adequate flow. However, the suction catheter has a preset and is specific for the anatomic site. This “fixed mode” does not balance the flow and vacuum requirements. The flexible tube, referred to as a suction catheter, has one or several holes at the end thereby allowing flow of fluid to a container outside the body. For example, too many holes will provide adequate flow, but the pressure differential “vacuum” may not be maintained; too few holes will maintain adequate vacuum but may not allow sufficient fluid flow.

Not having accurate control of the vacuum source can pull tissue into the hole leading to injury and or damage to the tissue. Bleeding, perforation, and death of tissue may ensue along with serious clinical harm. Accordingly, there is a need in the industry to mitigate tissue damage.

Prior inventions have approached the issue by limiting the time that the vacuum is applied to the suction catheter. Clinical standards call for 16 seconds of applied vacuum followed by an 8 second “off” period whereby the tissue is allowed to float away from the suction catheter. This “off” period has been determined to be necessary to avoid tissue damage.

Until now, timing of the on-off cycle has been accomplished using the available negative pressure from the vacuum source. A diaphragm-bellows is allowed to collapse under the negative pressure, and atmospheric pressure is bled into the bellows at a specified rate. Mechanical work is performed by the bellows, which opens and closes the regulated pressure to the patient. Timing of the on-off cycle is performed by varying the cross sectional area of the orifice that fills and empties the bellows. This leads to a rather inaccurate timing cycle, and one that either cannot be adjusted by the clinician, or if adjusted, is subject to large variation of timing as it tends to drift over time. Similar problems can also be found in a modular approach that uses a sandwich of plastic plates, air channels, springs and gaskets to achieve the same function as the bellows.

There is also need within the industry to create an alternative to the prior suction regulator, allowing for a low cost, accurate, electronic device that avoids the timing variations associated with traditional vacuum-timed regulators.

### BRIEF DESCRIPTION OF THE SEVERAL VIEWS OF THE DRAWINGS

The foregoing summary, as well as the following detailed description of the invention, will be better understood when read in conjunction with the appended drawings. For illustrating the invention, the figures are shown in the embodiments that are presently preferred. It should be understood, however, that the invention is not limited to the precise arrangements and instrumentalities shown. In the drawings:

FIG. 1 depicts at least one embodiment of the invention, namely a front-angular view of a suction regulator.

FIG. 2 depicts at least one embodiment of the invention, namely a back-angular view of a suction regulator.

FIG. 3 depicts at least one embodiment of the invention, namely a front-angular view of a suction regulator with the regulator cover removed.

FIG. 4 depicts at least one embodiment of the invention, namely a side, view of a suction regulator.

FIG. 5 depicts at least one embodiment of the invention, namely a front view of a suction regulator with the regulator cover and the vacuum gauge removed.

FIG. 6 depicts at least one embodiment of the invention, namely a front-angular view of a suction regulator with the regulator cover removed depicting the connection of the micro-latching solenoid valve and the main valve.

FIG. 7 depicts at least one embodiment of the invention, namely a front-angular view of a suction regulator with the regulator cover removed depicting the connection of the micro-latching solenoid valve, the main valve and the micro-controller.

FIG. 8 depicts at least one embodiment of the invention, namely a front-angular view of a suction regulator with the regulator cover removed depicting the removal of the connection of the micro-latching solenoid valve, the main valve and the micro-controller.

FIG. 9 depicts at least one embodiment of the invention, namely a front-angular view of a suction regulator with the regulator cover removed depicting the cross-sections of the micro-latching solenoid valve, and the main valve.

FIG. 10 depicts at least one embodiment of the invention, namely a cross-section of the main valve.

FIG. 11 depicts at least one embodiment of the invention, namely a cross-section of micro-latching solenoid valve.

FIG. 12 depicts at least one embodiment of the invention, namely the suction control system depicting the micro-latching solenoid valve, the main valve and the micro-controller.

FIG. 13 depicts at least one embodiment of the invention, namely the suction control system depicting the micro-latching solenoid valve, the main valve and the micro-controller.

FIG. 14 depicts at least one embodiment of the invention, namely the micro-latching solenoid valve.

FIG. 15 depicts at least one embodiment of the invention, namely the micro-latching solenoid valve.

FIG. 16 depicts at least one embodiment of the invention, namely the micro-latching solenoid valve.

FIG. 17 depicts at least one embodiment of the invention, namely the wireless suction control system.

### DESCRIPTION OF THE INVENTION

The present invention depicts an inventive solution to the fore mentioned issues related to suction regulators.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described, or referenced herein, are well understood and commonly employed using conventional methodology by those skilled in the art.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, “Vacuum” refers to the difference between atmospheric pressure and sub-atmospheric pressure, created by a vacuum-producing device such as a vacuum pump.

As used herein in the specification and in the claims, “gas,” or “air” means a compressible fluid such as oxygen, nitrogen, hydrogen, air (a mixture of dry air contains roughly (by volume) 78.09% nitrogen, 20.95% oxygen, 0.93% argon, 0.039% carbon dioxide, and small amounts of other gases. Air also contains a variable amount of water vapor, on average around 1%.), carbon dioxide, nitrous oxide, anesthetic and other similar gases or any combination thereof.

As used herein in the specification and in the claims, the term “commands” refers to; direct, instruct, call on, require, and control of an element over another.

As used herein in the specification and in the claims, the term “link” or “linked” refers to a connection, connector, coupling, joint or a relationship between two things or elements where one thing affects the other, both wireless, wired or in combination of both.

As used herein in the specification and in the claims, the term “transmit” or “transmits” refers to pass on at least one signal or information, in both digital or analog form, from one place or element to another both wireless, and wired or in combination of both.

The invention herein applies generally to General-purpose, Surgical and Tracheal suction regulators. In one embodiment of the invention, at least one micro-latching valve 401 is opened and closed by at least one solenoid electromechanical actuator. The micro-latching solenoid valve in turns opens and closes at least one main valve 402 that is connected the hospital vacuum or gas intake conduit. The latching nature of the solenoid, along with its low power activation allows for a battery powered, long-life device. Timing and control of the electromechanical actuator is performed by a low power micro-controller 304. This provides opportunity for highly accurate timing cycles, user adjustable timing intervals and feedback loop control operations.

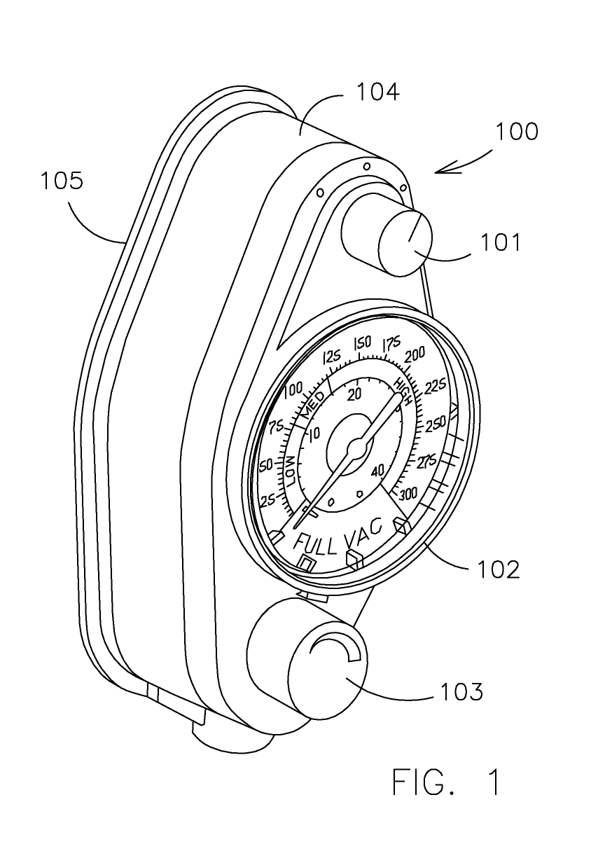

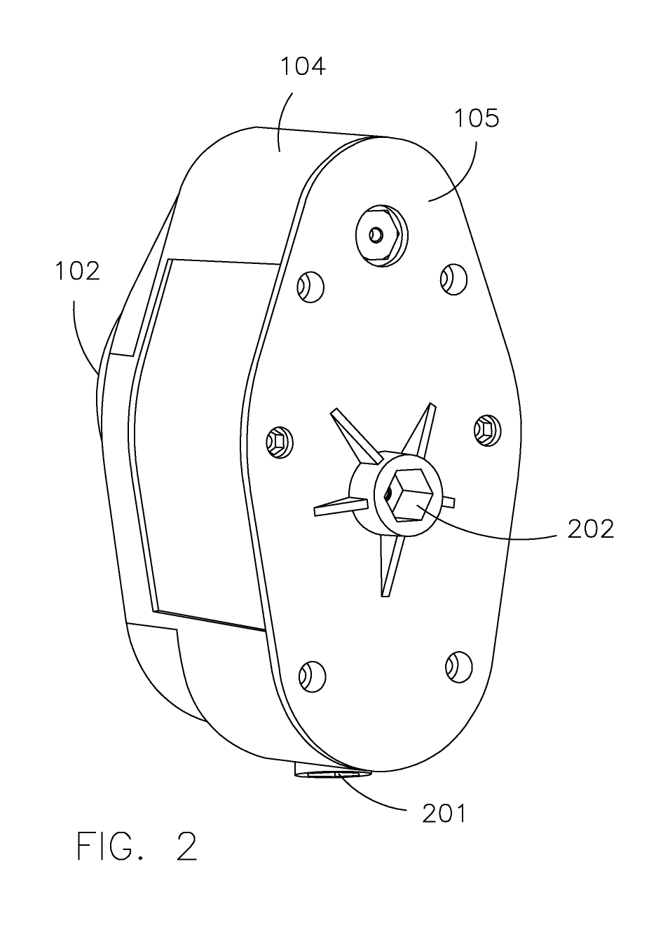

Referring now to the drawings in detail, in at least one embodiment of the invention, in FIG. 1, the suction regulator 100, comprises, the mode switch 101, the vacuum gauge 102, the manual air regulator 103, the regulator cover 104, and the conduit back-board 105. The vacuum gauge 102, in this embodiment is an analog gauge. FIG. 2, shows the backside of at least one embodiment of the invention. Here, the vacuum gauge 102, the regulator cover 104, the conduit back-board 105, the patient vacuum port 201, and the hospital vacuum intake port 202.

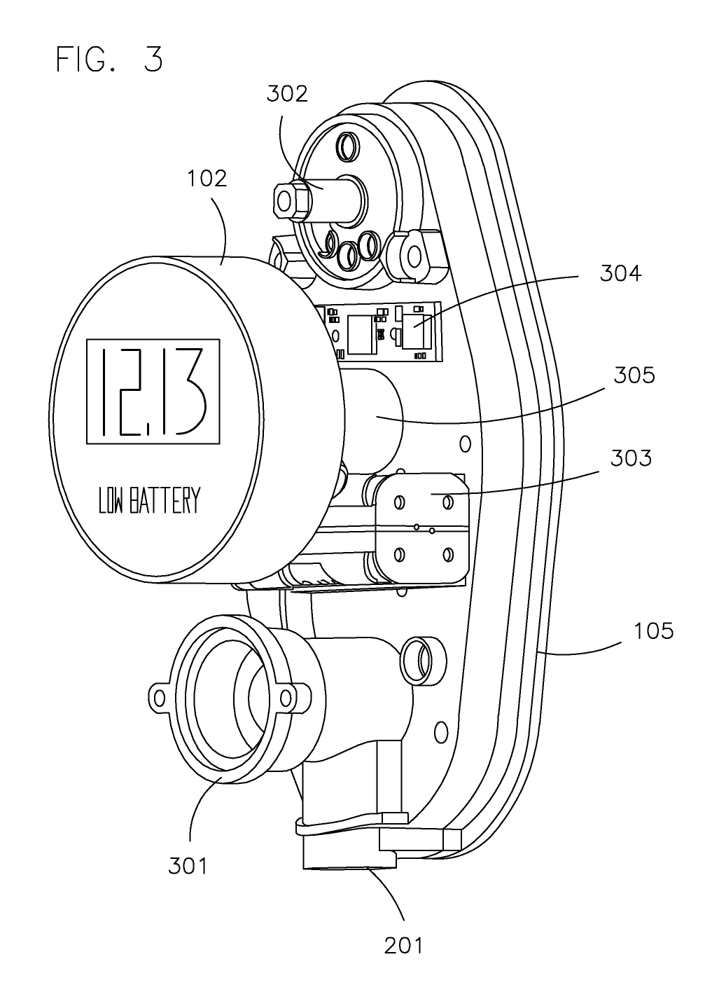

In another embodiment of the invention, FIG. 3 depicts the vacuum gauge 102, which in this embodiment is a digital gauge 102. The gauge 102 used reads vacuum from 760 torr to 0.001 torr using a sophisticated, microprocessor-based circuit to measure vacuum using a rugged, inexpensive, thermocouple vacuum gauge tube. It further depicts the mode switch connector 302, the micro-controller 304, the gauge connector 305, the power source 303, the conduit back-board 105, the patient vacuum port 201, and the manual air regulator connector 301.

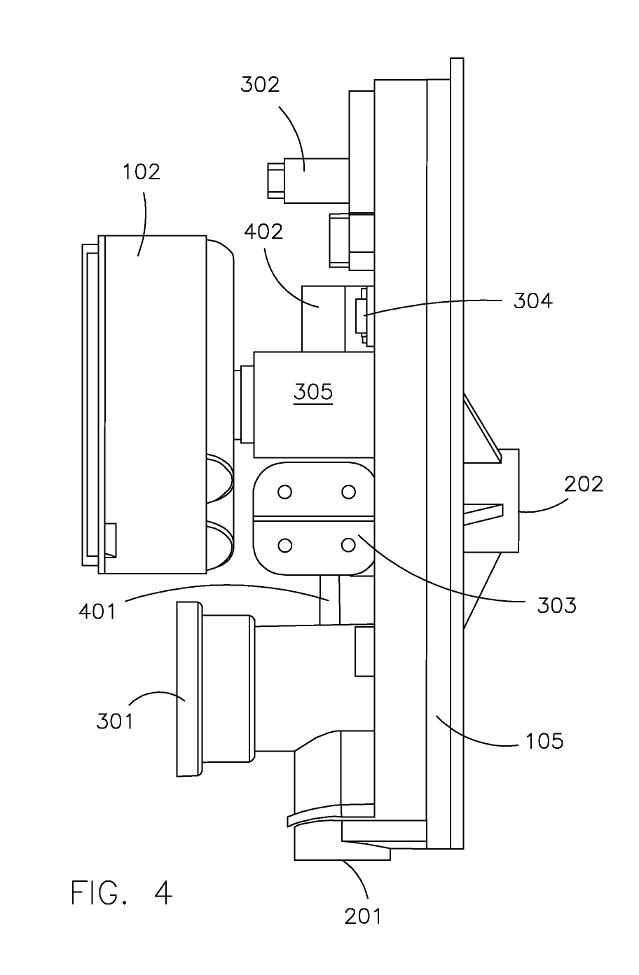

In a side view of one embodiment of the invention, FIG. 4 depicts the vacuum intake port 202, where the vacuum is connected to, usually a hospital wall inlet. The vacuum continues to the conduit back-board 105. The vacuum can be manually switched between intermittent, continuous or closed, at the mode switch connector 302. The vacuum is regulated using the manual gas regulator connector 301. The electrical pieces of the invention are; the micro-latching solenoid valve 401, the power source 303, the main valve 402, the micro-controller 304, and the digital vacuum gauge 102. The vacuum gauge 102 measures the vacuum through the gauge connector 305.

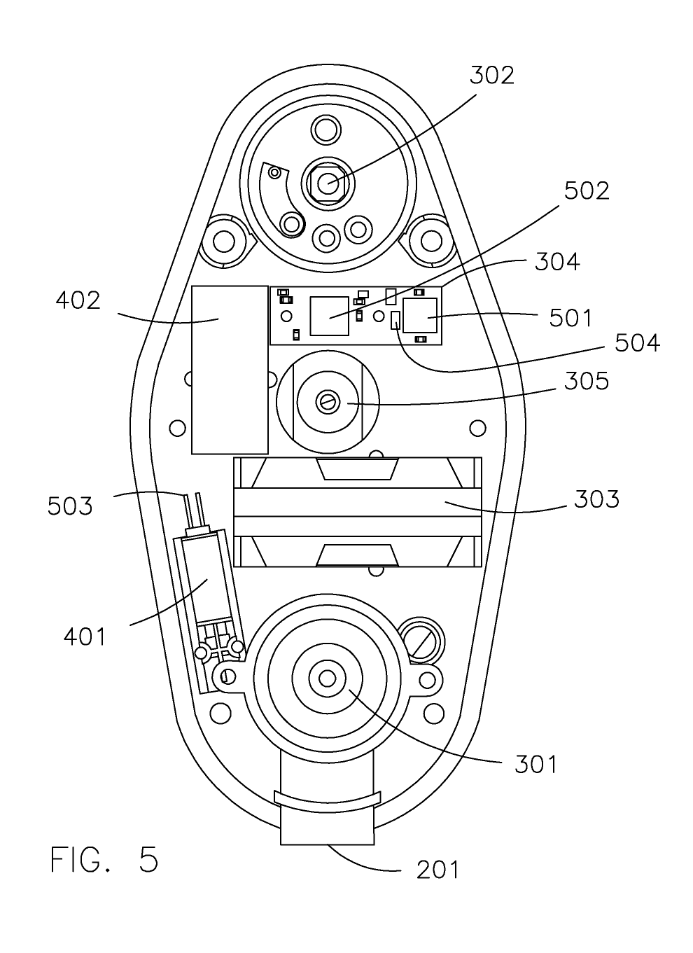

On a front view of one embodiment of the invention, after removing the vacuum gauge 102 from the gauge connector 305, FIG. 5 depicts the mode switch connector 302, the patient vacuum port 201, the power source 303, the micro-latching solenoid valve 401, the main valve 402, the micro-controller 304. The micro-controller 304 which further comprises the memory chip 501, the CPU 502, the wireless transmitter 504. A micro controller 304 can be considered a self-contained system with a processor, memory and peripherals and can be used as an embedded system.

While some self-contained micro-controller systems 304 are very sophisticated, many have minimal requirements for memory and program length, with no operating system, and low software complexity. Typical input and output devices include; switches, relays, solenoids, LEDs, small or custom LCD displays, radio frequency devices, and sensors for data such as flow, pressure, temperature, humidity, light level, etc. A self-contained system micro-controller 304 can be used in the same way for the same purpose to achieve the same result as a non-embedded system.

The micro-controller 304 should provide real time (predictable, though not necessarily fast) response to events in the flow system it is controlling. In the invention herein, response to the pressures to control the micro-latching solenoid valve 401 which in turn controls the main valve 402. The micro-controller 304 for this application, usually contains several to dozens of general purpose input/output pins (GPIO). GPIO pins are software configurable to either an input or an output state. When GPIO pins are configured to an input state, they are often used to read sensors such as or external signals (such as flow and pressure). Configured to the output state, GPIO pins can drive external devices such as the vacuum gauge 102 or the micro-latching solenoid valve 401.

The wireless transmitter 504 as used in this invention comprises wireless communications which can be via: radio frequency communication, microwave communication, short-range communication, infrared (IR) short-range communication with at least one of the purposes being point-to-point communication, point-to-multipoint communication, broadcasting, cellular networks and other wireless networks. The wireless transmitter 504 for this suction regulator 100 is embodied in a wireless local area network (WLAN) which links two or more suction regulators 100 over a short distance using a wireless distribution method, usually providing a connection through an access point for Internet access. The use of spread-spectrum or OFDM technologies allows the suction regulators 100 to move around within a local coverage perimeter, and still remain connected to the network. Products using the IEEE 802.11 WLAN standards are marketed under the Wi-Fi brand name. In another embodiment, the wireless transmitter 504 is a fixed wireless technology that implements point-to-point links between suction regulators 100 or networks at two distant locations, often using dedicated microwave or BLUETOOTH® signals.

In one embodiment of this invention, the power source 303, is at least one lithium-ion battery. Although a DC or AC cable attached to the device 100, would work in the same way to achieve the same function and give the same result as a battery powered suction regulator 100. In this embodiment, a (lithium-manganese dioxide) LiMnO2 was used. This type of battery was chosen because the suction regulator 100 requires long shelf life and the selected battery has a very low rate of self discharge, usually around 10 years. A lithium-ion battery (sometimes Li-ion battery or LIB) is a family of rechargeable battery types in which lithium ions move from the negative electrode to the positive electrode during discharge, and back when charging. Any other type of chemistry in the power source 303 can be used in the same way to accomplish the same result, which is to move a micro-latching solenoid valve 401 typically around 5 milliwatts per actuation.

In another embodiment of the invention, FIG. 6 depicts how the micro-latching solenoid valve 401 and the main valve 402 is interconnected. Here the normally closed conduit 701 links the conduit back-board 105. The common conduit 702, links the micro-latching solenoid valve 401 to the main valve 402. The normally open conduit 703 is open to the atmosphere. Here the micro-latching valve connector 503 is depicted along with the mode switch connector 302, the manual air regulator connector 301, the mode switch connector 302, the power source 303, the micro-controller 304, the gauge connector 305, the micro-latching solenoid valve 401, the main valve 402, the memory chip 501, the CPU 502, the wireless transmitter 504, and the patient vacuum port 201.

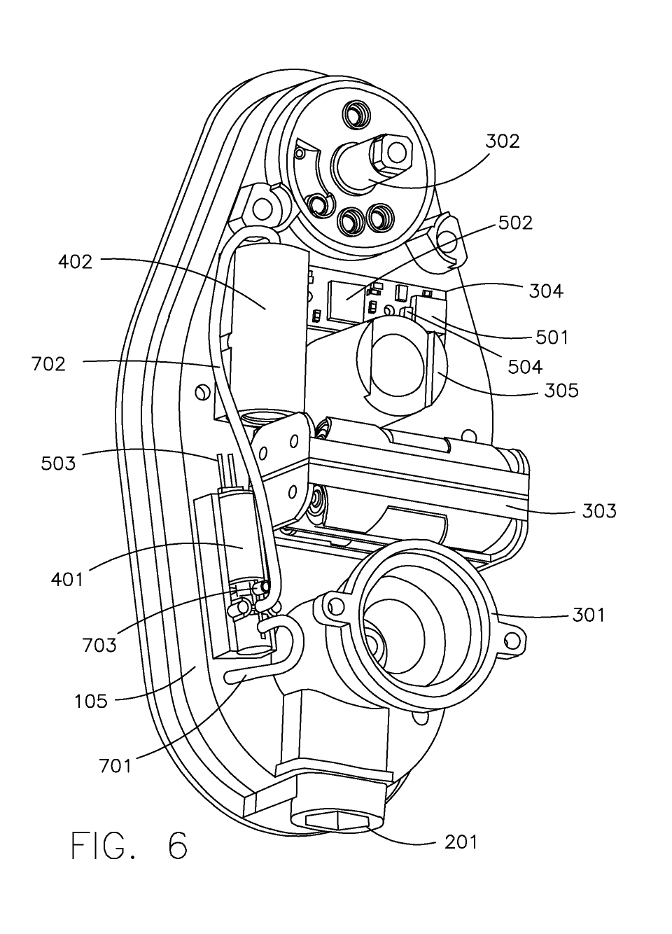

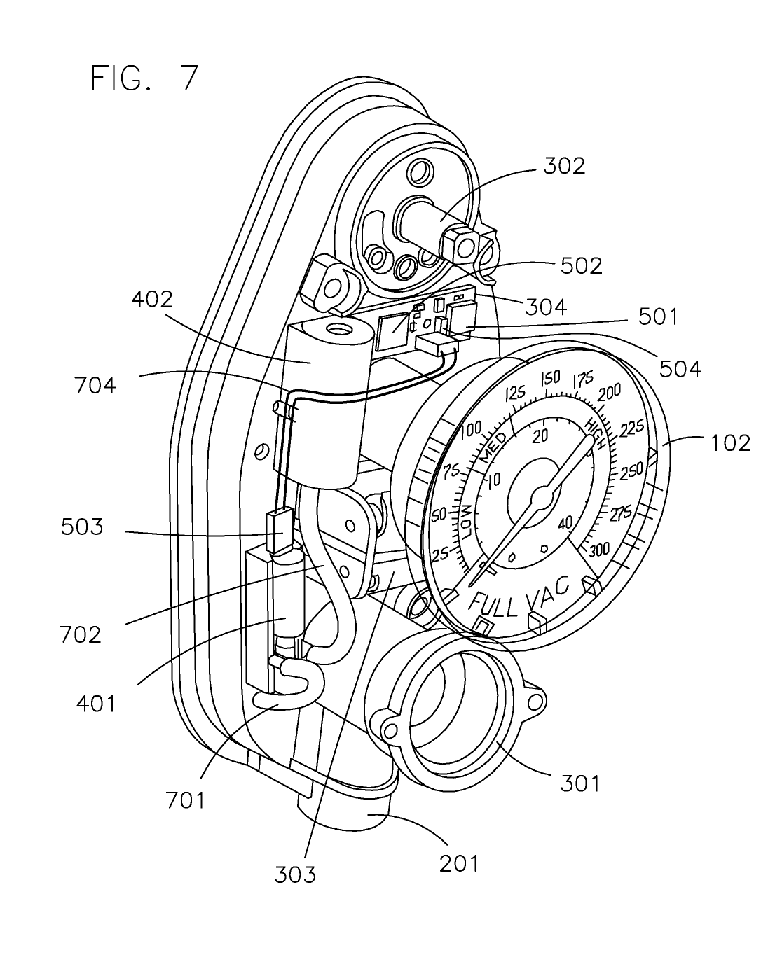

In yet another embodiment of the invention, FIG. 7 depicts how the micro-latching solenoid valve 401 is electrically connected to the micro-controller 304 by solenoid valve electrical connection 704. Here, the vacuum gauge 102 is analog. The common conduit 702 that links the micro-latching solenoid valve 401 to the main valve 402 is shorter and is placed on the bottom of the main valve 402.

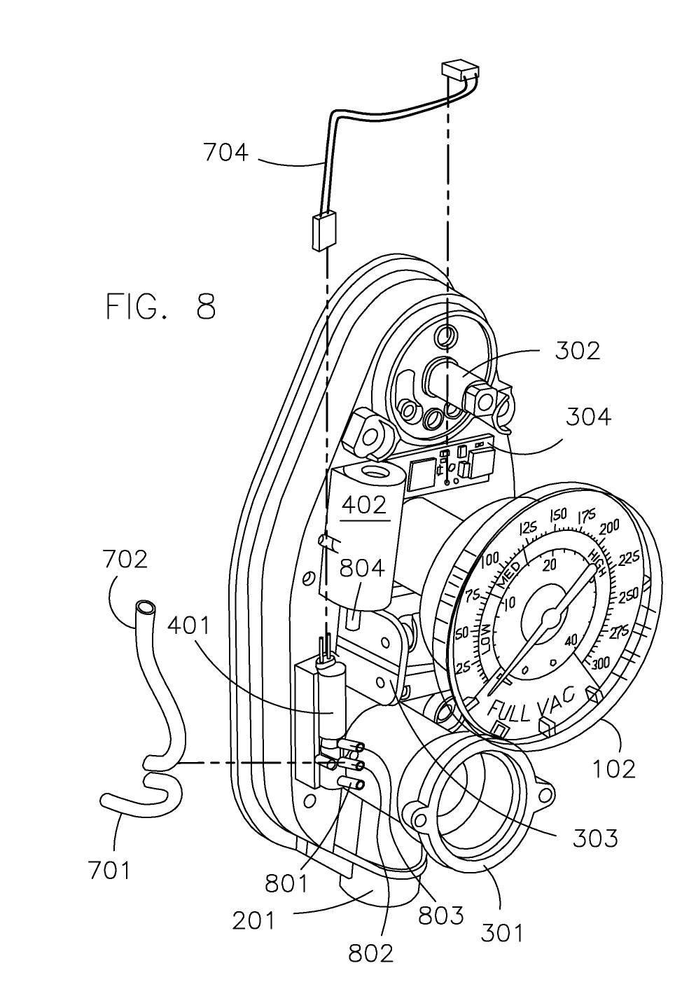

FIG. 8 depicts how the suction regulator 100 looks when regulator is in the following states; the normally closed conduit 701, common conduit 702, and the solenoid valve electrical connection 704. Here, the suction regulator 100, comprises, at least one micro-latching solenoid valve 401, said at least one micro-latching solenoid valve 401 further comprise at least one solenoid electromechanical actuator 1107, at least one main valve 402, said at least one main valve 402 further comprising at least one main valve spring 1002, said at least one main valve 402 is attached to a vacuum source via the patient vacuum port 201, and regulated with the manual air regulator connector 301.

The at least one micro-controller 304 commands said at least one solenoid electromechanical actuator 1107, and at least on power source 303, said at least one power source 303 provides power to said at least one electromechanical actuator 1107 and said at least one micro-controller 304, wherein the at least one micro-latching solenoid valve 401 controls the flow of vacuum through the at least one main valve 402. The suction regulator 100, further comprises, a vacuum gauge 102, a mode switch 302, a manual air regulator 301, a patient vacuum port 201, and at least one flow sensor 1201.

FIG. 8 exposes the micro-latching solenoid valve 401 after the normally closed conduit 701, and common conduit 702 are removed. The three solenoid valve openings are revealed; namely, the normally closed conduit connector 801, the common conduit connector 802, and the normally open conduit connector 803. In this embodiment all three connectors are facing up, but in other embodiments, at least one can be linked to the conduit back-board 105 where channels in said board further link the micro-latching solenoid valve 401 to the vacuum source. Here the main valve common conduit connector 804 is also exposed.

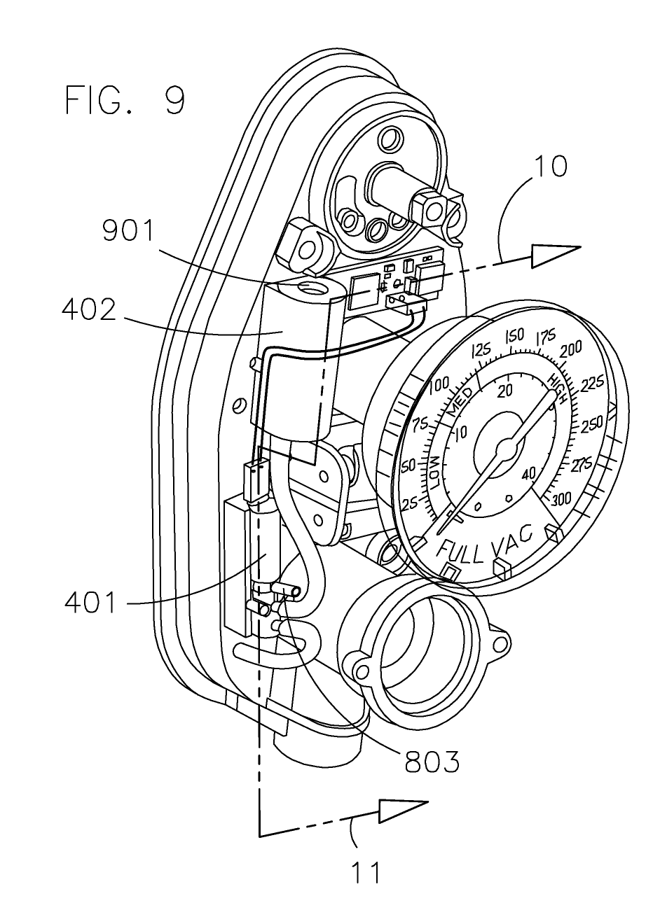

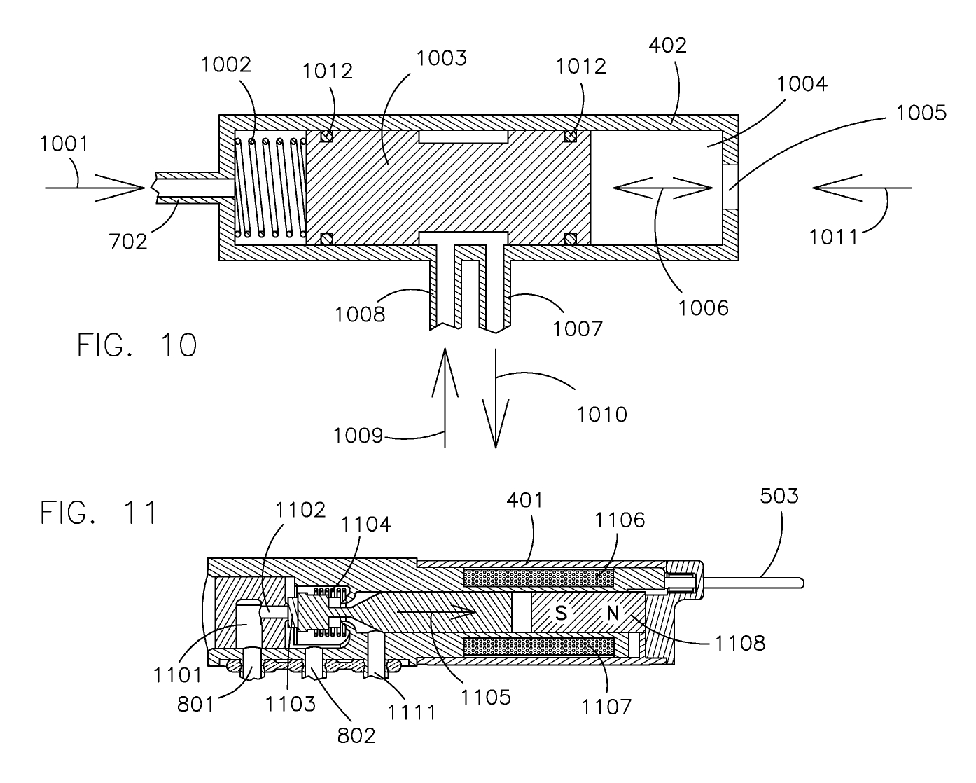

FIG. 9 depicts the openings exposed to the atmospheric pressure, namely valve atmospheric entrance port 901, and the normally open conduit connector 803. Here the main valve 402 is cut in to FIG. 10 and the micro-latching solenoid valve 401 will be cut into FIG. 11.

In one embodiment of the invention, FIG. 10 depicts a cross-section of the main valve 402. Here the main valve common conduit connector 804 is depicted. Said main valve common conduit connector 804 joins the micro-latching solenoid valve 401. The micro-latching solenoid valve 401 in its open position allows for vacuum 1001 to pull the sliding seal body 1003 to push against main valve spring 1002. When the micro-latching solenoid valve 401 is in it's closed position, the main valve spring 1002 pushes back on to the sliding seal body 1003. In order to prevent leakage of air, a couple of “O” rings 1002 are placed to seal the sliding seal body 1003. The sliding seal movement 1006, opens and closes the patient port 201 and the port vacuum source 202. Air direction from patient 1009 is blocked or allowed to pass in the direction to vacuum source 1010. As the sliding seal body 1003 is pulled against the main valve spring 1002, atmospheric air 1011 is pulled in to the valve chamber 1004 though valve atmospheric entrance port 901.

The sliding seal body 1003 as used herein comprises embodiments in a variety of valve types, such as the ones used in the automatic control of air, gases and other industrial compressible fluids. These include valve types which have linear and rotary spindle movement. Linear types include globe valves, sliding membrane seal, slide valves and bellows. Rotary types include ball valves, butterfly valves, plug valves and their variants. All of them can be used in the same way, for the same function to achieve the result of opening and closing the vacuum source port to vacuum 202 from the patient at the port to patient 201.

In one embodiment of the invention, FIG. 11 depicts a cross-section of the micro-latching solenoid valve 401 and the solenoid electromechanical actuator 1107. Here the micro-latching solenoid valve chamber 1101, the micro-latching solenoid valve conduit 1102, the micro-latching solenoid valve 1103, the micro-latching solenoid valve spring 1104, the solenoid actuator coil 1106, the solenoid electromechanical actuator 1107 and the permanent magnet 1108 are depicted. Depicted in FIG. 11 are also; the micro-latching valve connector 503, the normally closed conduit connector 801, the common conduit connector 802, and the normally open conduit connector 803.

The solenoid electromechanical actuator 1107 is drawn in by the vacuum though the normally closed conduit connector 801. It is normally closed because the source vacuum forces the micro-latching solenoid valve 1103 to close against the micro-latching solenoid valve conduit 1102. When a pulse of electricity is fed into micro-latching valve connector 503 and goes to the solenoid actuator coil 1106, the end of the solenoid electromechanical actuator 1107 is magnetized negative, thereby attracting to permanent magnet 1108. The micro-latching solenoid valve is moved in direction 1105, thereby opening the micro-latching solenoid valve conduit 1102 normally closed by the source vacuum, or micro-latching solenoid valve spring 1104.

The latching nature of the solenoid valve 401 avoids the power requirements of a standard solenoid. A 10 millisecond pulse of current moves the solenoid 1107 to one extreme of displacement where it stays in the location without additional power. In an alternative embodiment, a similar pulse of opposite polarity will actuate the solenoid 1107 to its alternative position, again without the need of continuous power.

The solenoid such as the one depicted in FIG. 11 can control the flow of air by: 1) A main valve 402 action whereas the solenoid 1107 opens or closes an orifice 1102, or 2) locking and unlocking a mechanical slide or valve that directly opens or closes an orifice 1102, or 3) open a small “pilot” hole 1102 that bleeds pressure from a larger reservoir. This allows for accurate control of the vacuum source and avoids the pull of tissue into the vacuum tube leading to injury and or damage to patient or tissue.

FIG. 12 in general, illustrates how a large valve 401 can be controlled by actuating a small solenoid, 1107 in the micro-latching solenoid valve 401. The main valve, 401, slides left or right for the inlet 201 and outlet ports 202 to allow flow of a vacuum 1009 and 1010. When not activated, the spring 1002, pushes the valve body 1003, to the right or to the left, closing or opening the inlet 201 and outlet ports 202.

In detail, a pulse of electricity is generated by the micro-controller 304. This pulse, is received by micro-latching valve connector 503, which in turn passes electricity to the solenoid actuator coil 1106, which in turn magnetizes the solenoid electromechanical actuator 1107 negative, thereby attracting to permanent magnet 1108. This electromagnetically induced movement overcomes the source vacuum from normally closed conduit 701. Hence, this attraction causes a movement that opens micro-latching solenoid valve 1103 to allow the flow of vacuum to pass from common conduit 702 linked to the main valve 402 to the normally closed conduit 701. By opening micro-latching solenoid valve conduit 1102 vacuum forces sliding seal body 1003 to move against a position set by main valve spring 1002 widening the valve chamber 1004 allowing atmospheric air 1011 to enter through valve atmospheric entrance port 901. Atmospheric pressure 1011 on the right side of the valve body overcomes the spring pressure 1002, and the valve body slides to the left.

The ‘O’ rings 1012 illustrated in the FIG. 12, may be replaced by sliding membrane seals, whichever is easier to assemble. The main valve 402, can also be replaced by a person skilled in the art with, a globe valve, a sliding membrane seal, a slide valve, a bellows, a ball valve, a butterfly valve, a plug valve and any combination thereof.

In one embodiment of the invention, FIG. 12 also depicts a suction control system 100, comprising; the managing of the flow of vacuum 1009 and 1010 in at least one main valve 402 by using at least one flow sensor 1201, 1202 and at least one micro controller 304 to create a feedback loop 1203, 1204, and activating at least one electromechanical actuator 1107 in at least one micro-latching solenoid valve 401 using said feedback loops 1203, 1204, wherein said at least one micro-latching solenoid valve 401 commands said at least one main valve 402. The suction control system 100, wherein the said at least one micro-controller 304 further comprises at least one CPU 502, at least one wireless transmitter 504, and at least one memory chip 501. The said at least one micro-controller 304 allows for timing cycles, user adjustable timing intervals and feedback loop control operations. Accurate user adjustable timing is now ingeniously created by the invention herein, allowing accurate suction intervals to the patient and avoiding the problems of current vacuum regulators.

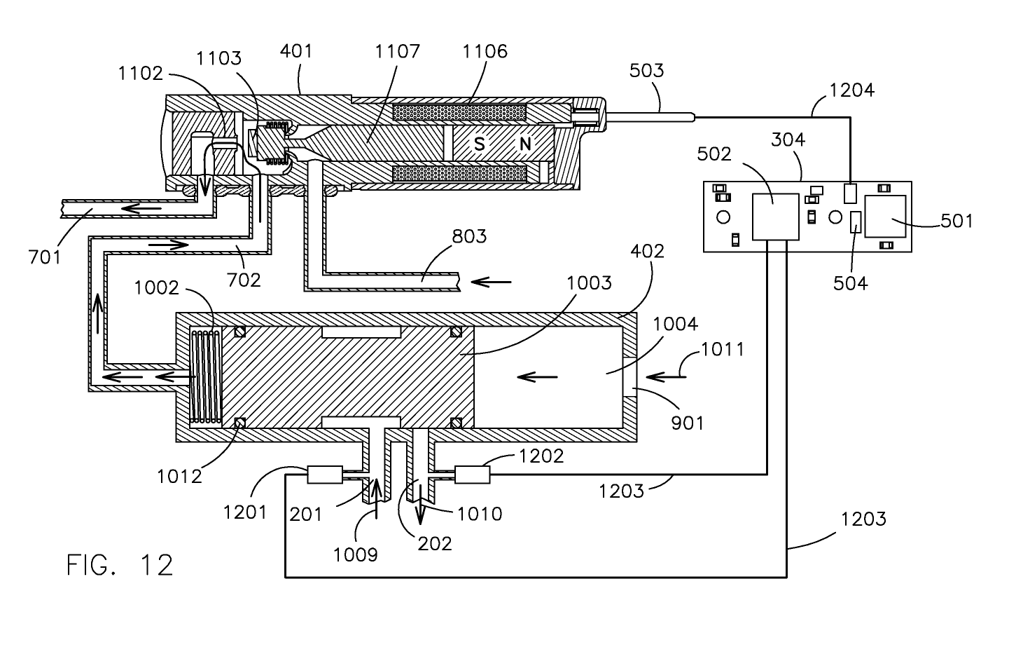

FIG. 13 illustrates how the suction control system 100, works when the micro-latching solenoid valve 401 is in its closed position. Here, no pulse of electricity is generated by the micro-controller 304. No electricity passes to the solenoid actuator coil 1106 by the micro-latching valve connector 503, which in turn de-magnetizes the solenoid electromechanical actuator 1107 (now positive), thereby repulsing from permanent magnet 1108. This repulsion along with the suction force from the hospital vacuum in the normally closed conduit 701 closes micro-latching solenoid valve 1103 to prevent the flow of vacuum from common conduit 702 linked to the main valve 402 to the normally closed conduit 701. Simultaneously, the atmospheric air enters through the normally open conduit connector 803 to the common conduit 702 and releases the sliding seal body 1003 to move to the position set by main valve spring 1002 closing the valve chamber 1004 allowing atmospheric air 1011 exit through valve atmospheric entrance port 901.

Atmospheric pressure 1011 on the right side of the valve body overcomes the spring pressure 1002, and the valve body slides to the left. A person skilled in the art can calculate the forces needed to open and close the main valve 402 by measuring the diameter of the main valve 402, and the spring constant, 1002, and the pressure applied to both sides of the sliding valve body 1003.

When the sliding valve body 1003 opens or closes the port to source vacuum 202 and the port to patient 201, flow of vacuum from the vacuum direction from patient 1009 vacuum direction to source vacuum 1010 is controlled. This control is attributed to the flow sensors 1201 and 1202 who in turn sends a signal of the amount of flow in both the source vacuum intake port 202 and the patient vacuum port 201. This information is transformed, stored analyzed or compared to pre-set ranges in the micro-controller 304 which in turns send more or less electrical pulses via feed back loop 1204 to the micro-latching solenoid valve 401 to open or close the flow of vacuum to the main valve 402. This “off” period has been determined by the micro-controller 304 to be necessary to avoid any tissue damage.

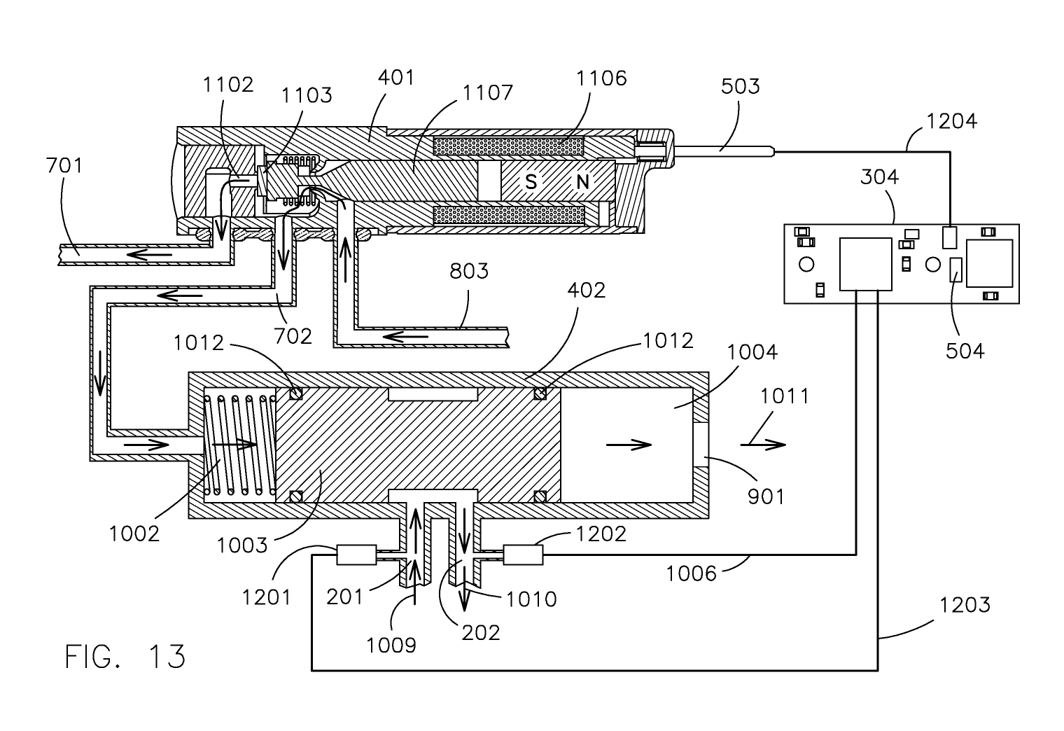

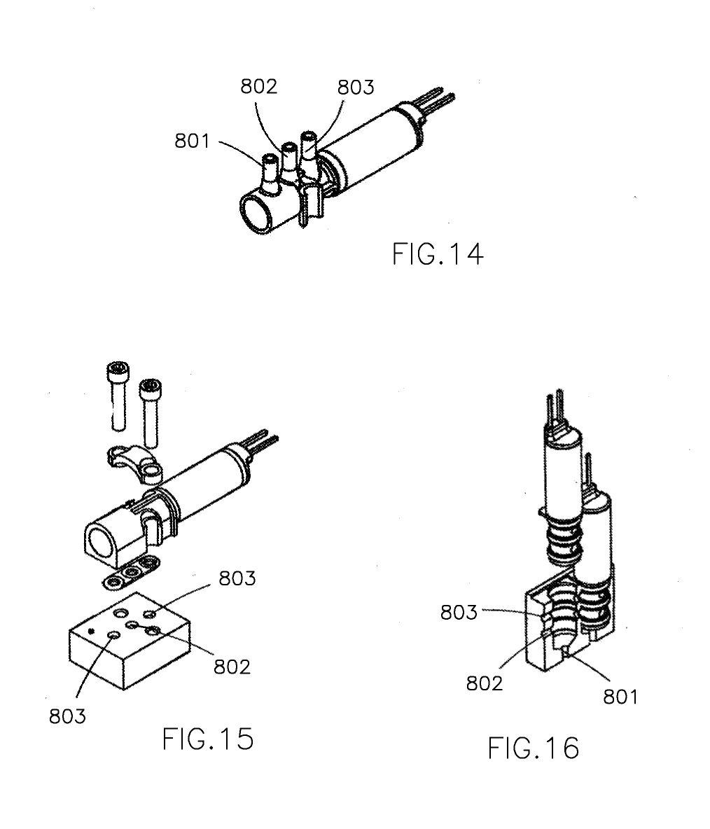

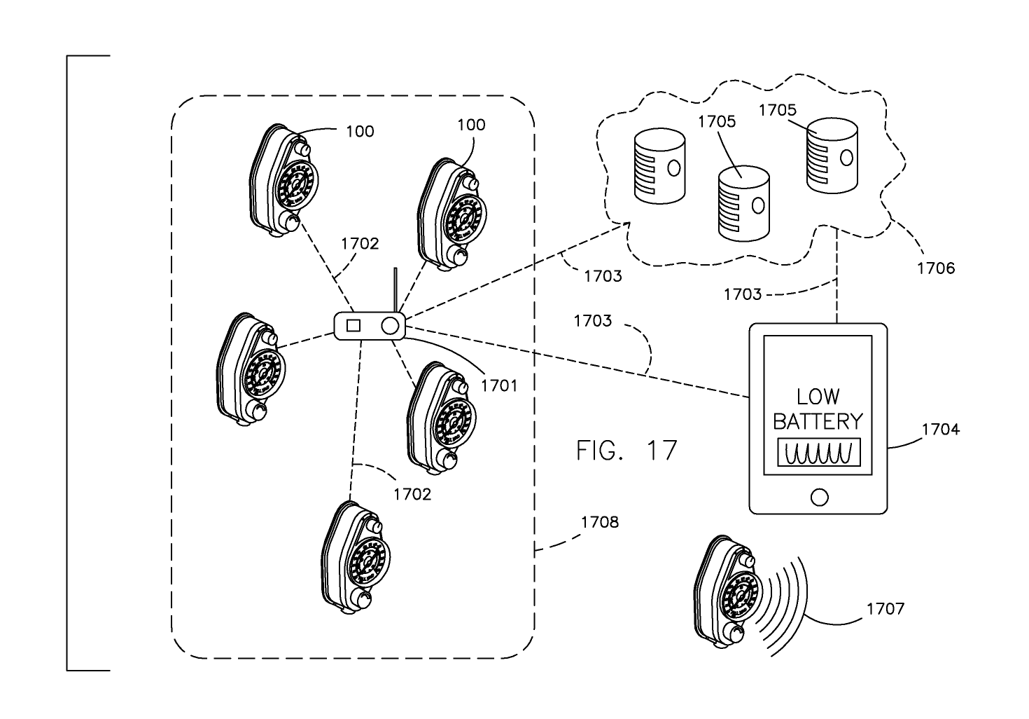

FIG. 14, FIG. 15 and FIG. 16 depicts different embodiments of the micro-latching solenoid valve 401 that can be used in the same way for the same purpose to accomplish the same result as previously described. Here, all solenoid valves comprise the same elements, namely, the normally closed conduit connector 801, the common conduit connector 802, and the normally open conduit connector 803. A typical High Density Inter-face (HDI) solenoid valve as the one used in the invention herein, offers more flow capacity without sacrificing size and weight. A HDI solenoid valve provides the features of a large valve coupled with the superior performance of a miniature valve in one compact design. A spike and hold voltage drive is used to achieve an extended flow and pressure range. Typical specification comprise Compact Size Light Weight: Less than 4.5 grams Operating Pressure Range: Vac-50 psig (0-50 psid), Spike and Hold Voltage Drive Required, Electrical Connection: 0.025″ Sq. Pin, Wetted Materials: PPA, PBT, 316SS, 430F SS, FKM, Epoxy, Operating Temperature Range: 40° F. to 120° F.

FIG. 17, depicts at least one embodiment of the invention, namely a wireless suction control system of many suction regulators 100 comprising; a network 1702, said network 1702 further comprising, a plurality of suction regulators 100. Wherein the said plurality of wireless suction regulators 100 are linked to at least one router 1701 with said at least one wireless transmitter 504, and said network 1702 is linked through said at least one router 1701 to the internet 1706.

At least one of the purposes of the mesh network 1702 is to provide a visual landscape to persons responsible for the proper function and maintenance of medical devices within a health care setting using a wireless device or monitoring station 1704 or be monitored at a manufacturing facility via an internet connection 1703. The wireless mesh network 1702 can display device specific information or provide information on device movement/location.

The visual landscape example is depicted in FIG. 17. This visual information is served to an electronic device or handheld computer or monitoring station 1704 that includes an electronic display, a microprocessor, and wireless connectivity. In addition to the mesh network created by the population of devices 100 there are also fixed transceivers found within a set perimeter 1708 or along the perimeter 1708. As a tool that determines location in real or near-real time, the wireless mesh network 1702 offers a loss-prevention function. By establishing a perimeter 1708 or maximum allowable range users can be notified when a device 100 is on the move or moves beyond a defined perimeter 1708. If a device is outside the perimeter 1708, the suction regulators 100 will produce an alarm.

The device specific information that can be provided wirelessly from the device 100 to the user at at least one monitoring station 1704 might include; vacuum source pressure, battery life, location in relation to other devices, date when maintenance was last performed, next maintenance due date, repair history, ambient temperature, location in relation to other devices etc. All this information can be fed wirelessly 1703 by the router 1701 to the internet 1705 were the information can be stored in servers 1705 for later retrieval, analysis and monitoring.

Each wall suction regulator 100 is equipped with and utilizes appropriate control circuitry such that each unit is part of a mesh network 1702, providing communication either via wire, fiber optic, radio signal, or light signal 1702 between units 100. Such mesh network enables all of the units (an array) either within a physical plant (local area network) or outside of a physical plant (wide area network) to communicate with each other.

Software revisions can be sent via wire, light, or radio to a single unit 100, and this unit 100 can pass the software revision to each successive unit within the array, be it in a local area network within a structure or outside of the structure in a wide area network. Battery status, working status, temperature, time of operation, out of range alarm, wall suction pressure, hospital infrastructure pressure, etc., may be sent along the mesh network to a plurality of central monitoring stations 1704 within a local area network 1702 or wide area network 1703, such that all of these parameters can be monitored even though the unit 100 of interest within the array of units is outside of the radio range 1708 of the monitoring station 1704.

In yet another embodiment of the invention, the mesh network 1708 can be built upon an array connected via wire, radio signal, light signal, fiber optic signal, etc. Each unit 100 within the array has a unique electronic address. There is may or may not be a master unit, and each unit 100 is identical. Hence, each unit in the array may assume a control function if deemed necessary by the programmer, although a master unit is not necessary for the mesh network to function properly.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

It is to be appreciated that the Detailed Description section, and not the Abstract section, is intended to be used to interpret the claims. The Abstract section may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. The breadth and scope of the present invention should not be limited by any of the above-described exemplary embodiments, but should be defined only in accordance with the following claims and their equivalents.

We claim:

1. A suction regulator, comprising:
at least one micro-latching solenoid valve, said at least one micro-latching solenoid valve further comprising at least one electromechanical actuator;
at least one main valve, said at least one main valve further comprising at least one main valve spring, and at least one sliding seal body, said at least one main valve is attached to a vacuum source;
at least one micro-controller, said at least one micro-controller commands said at least one electromechanical actuator; and
at least one power source, said at least one power source provides power to said at least one electromechanical actuator and said at least one micro-controller,
wherein the at least one micro-latching solenoid valve controls the flow of vacuum through the at least one main valve.

2. The suction regulator of claim 1, wherein the suction regulator further comprises, a regulator cover, a vacuum gauge, a mode switch, a manual air regulator, a hospital vacuum intake port, a patient vacuum port, at least one flow sensor, and a conduit back-board.

3. The suction regulator of claim 1, wherein the said at least one micro-controller along with said at least one flow sensor provides for a feedback loop control operation to said main valve.

4. The suction regulator of claim 1, wherein the said at least one micro-controller further comprises at least one CPU, and at least one memory chip.

5. The suction regulator of claim 1, wherein the said at least one main valve further comprises at least one “o” ring.

6. The suction control system of claim 1, wherein the said at least one sliding seal body, comprises, a globe valve, a sliding membrane seal, slide valve bellows, a ball valve, a butterfly valve, a plug valve, diaphragm-bellows, sandwich of plastic plates with air channels, springs and gaskets, and any combination thereof.

7. The suction regulator of claim 1, wherein the said at least one power source is a battery.

8. The suction regulator of claim 1, wherein the said at least one micro-controller allows for timing cycles, user adjustable timing intervals and feedback loop control operations.

9. A suction control system, comprising:
managing the flow of vacuum flow in at least one main valve by using at least one flow sensor and at least one micro controller to create a feedback loop; and
activating at least one electromechanical actuator in at least one micro-latching solenoid valve using said feedback loop,
wherein said at least one micro-latching solenoid valve commands said at least one main valve.

10. The suction control system of claim 9, wherein the said at least one micro-controller further comprises at least one CPU, at least one wireless transmitter, and at least one memory chip.

11. The suction control system of claim 9, wherein the said at least one main valve further comprises at least one “o” ring and at least one sliding seal body.

12. The suction control system of claim 11, wherein the said at least one sliding seal body comprises, a globe valve, a sliding membrane seal, a slide valve bellows, a ball valve, a butterfly valve, a plug valve, diaphragm-bellows, sandwich of plastic plates with air channels, springs and gaskets, and any combination thereof.

13. The suction regulator of claim 9, wherein the said at least one micro-controller allows for timing cycles, user adjustable timing intervals and feedback loop control operations.

14. A wireless suction control system, comprising:
a network, said network further comprising, a plurality of suction regulators, said plurality of suction regulators further comprising;
at least one micro-latching solenoid valve,
at least one main valve; and
at least one micro-controller, said at least one micro-controller further comprising at least one CPU, at least one wireless transmitter and at least one memory chip,
wherein the said plurality of wireless suction regulators are linked to at least one router with said at least one wireless transmitter, and said network is linked through said at least one router to the internet.

15. The suction control system of claim 14, wherein the said at least one micro-latching solenoid valve further comprises at least one electromechanical actuator.

16. The suction control system of claim 14, wherein said at least one micro-controller commands said at least one electromechanical actuator with a feed back loop.

17. The suction control system of claim 14, wherein the said at least one main valve further comprises at least one “o” ring and at least one sliding seal body.

18. The suction control system of claim 17, wherein the said at least one sliding seal body consists essentially of; a globe valve, a sliding membrane seal, a slide valve bellows, a ball valve, a butterfly valve, a plug valve, diaphragm-bellows, sandwich of plastic plates with air channels, springs and gaskets, and any combination thereof.

19. The suction control system of claim 14, wherein the plurality suction regulators further comprise, a vacuum gauge, a mode switch, a manual air regulator, a hospital vacuum intake port, a patient vacuum port, at least one flow sensor, and a conduit back-board.

20. The suction control system of claim 14, wherein the wireless transmitter transmits, battery status, working status, temperature, time of operation, out of range alarm, wall suction pressure, hospital infrastructure pressure, to at least one central monitoring station.
